(19) United States
(12) Patent Application Publication
(10) Pub. No.: US 2024/0148969 A1
(43) Pub. Date: May 9, 2024

(54) PORTABLE INFUSION PUMP WITH PINCH/SQUEEZE PUMPING ACTION

(71) Applicant: Modular Medical, Inc., Escondido, CA (US)

(72) Inventors: Paul M. DiPerna, Escondido, CA (US); Marc D. Goldman, San Diego, CA (US)

(21) Appl. No.: 18/412,509

(22) Filed: Jan. 13, 2024

Related U.S. Application Data
(63) Continuation-in-part of application No. 17/001,330, filed on Aug. 24, 2020, now abandoned.

(51) Int. Cl. A61M 5/168; A61M 5/142; A61M 5/14

(52) U.S. Cl. CPC A61M 5/16886; A61M 5/142; A61M 2005/1401; A61M 2205/0216; A61M 2205/10; A61M 2205/3327; A61M 2205/3334; A61M 2207/00

(57) ABSTRACT

A monitor for measuring the flow rate of a fluid medicament is incorporated into an elastomeric tube that will be used to transfer fluid medicament from a supply source to the patient. Structurally, the monitor includes a hollow shell having a flexible membrane that is affixed across the inside the shell to create a fluid tight barrier which bifurcates the shell's interior into a gas enclosure and a liquid pathway. As fluid passes through the liquid pathway from the supply source and on to the patient, movements of the flexible membrane cyclically indicate changes in the volume of the gas enclosure. Volume changes of the gas enclosure are measured by a pressure gage to indicate the flow rate of the fluid medicament.

### REFERENCE TO RELATED APPLICATIONS

This application is a continuation-in-part of application Ser. No. 17/001,330 filed Aug. 24, 2020, which is currently pending. The contents of application Ser. No. 17/001,330 are incorporated herein by reference.

### FIELD OF THE INVENTION

The present invention pertains to gas pressure monitors which can be used to continuously measure liquid flow rates being generated by a medical device. More specifically, the present invention pertains to monitors of gas pressure measurements that are commensurate with, but separated from, liquid volume changes which occur as the liquid is moved through a common bi-fluid chamber. The present invention is particularly, but not exclusively, useful for monitoring the flow of a liquid medicament through a portable medical medicament infusion system.

### BACKGROUND OF THE INVENTION

A primary operational concern for portable pumps which are to be used for infusing fluid medicaments to a patient is that they must have accurate fluid flow rates. Other desirable attributes for such a device include a structure for the pump that enhances safety, is comfortable for the patient, is easy to operate and is preferably, un-intrusive. Moreover, because infusion pumps are typically used continuously for extended periods of time, it is essential that the pump, or individual components thereof, be periodically removed from the patient for cleaning and minor maintenance, or to otherwise be replaced.

The work required to operate a liquid infusion pump typically involves the application of pressure forces on a predetermined volume of the liquid. Although there is a plethora of well-known liquid pumping mechanisms, the import of the present invention is directed to the capability of a pressure monitor for measuring volumetric liquid flow rates for medical applications. It is also well-known that pump components may be subject to random operational variations and sometimes experience a loss of operational efficacy over time. For these reasons, a pressure monitor can be useful for monitoring the volume of administered medications as well as anticipating a pump's decline in operational efficacy. Finally, pump components may fail, giving rise to safety concerns.

With the above in mind, it is an object of the present invention to provide a monitor for a portable infusion pump that is continuously operable to give accurate liquid flow rate measurements. Another object of the present invention is to provide a method for manufacturing such a monitor. Yet another object of the present invention is to continuously assess the operational capability of an infusion pump. Still another object of the present invention is to provide a monitor for a portable infusion pump that is safer, relatively simple to manufacture, is easy to use, and is comparatively cost effective.

### SUMMARY OF THE INVENTION

In accordance with the present invention a portable fluid flow monitor is provided which measures flow rates continuously with reliable accuracy. As intended for the present invention, the fluid flow monitor is employed in combination with a source of a liquid medicament and a pump, preferably a pump using a pinch/squeeze mechanism. In this combination, the fluid flow monitor is positioned between, and is respectively connected in fluid communication with the source of liquid medicament and with the pinch/squeeze mechanism.

Structurally, the fluid flow monitor is a hollow container having a rigid shell which surrounds a bi-fluid chamber. A flexible membrane is affixed to the shell inside the bi-fluid chamber with a fluid tight seal, to thereby separate the bi-fluid chamber into a gas enclosure and a liquid pathway. Together the gas enclosure and the liquid pathway establish a fixed combined volume V_{c} inside the hollow container.

The hollow container includes an input port that is formed on the shell to establish liquid access into the liquid pathway in the bi-fluid chamber. Functionally, the input port is adapted to selectively connect a contractible bag containing a volume, V_{b}, of liquid in fluid communication with the liquid pathway of the hollow container. With this connection, a transfer valve is provided for the input port which is adapted to alternatively establish a closed/open configuration for the input port in its communication with the contractible bag. Note: the contractible bag contains a volume, V_{b}, of liquid to be infused over an extended time. Thus, V_{b} is much larger than V_{c}.

In addition to its input port, the hollow container also includes an output port that is formed on the shell to alternatingly establish liquid communication between the liquid pathway and an elastomeric tube. Operationally, both the input port and the output port are adapted to alternatingly have open/closed configurations. In a preferred embodiment, configurations for the input port and the output port are controlled to ensure that when one port is open, the other port is closed. For this purpose, a control unit is connected to the input port and to the output port. With this connection the control unit is responsive to the open/closed configuration of the output port to thereby adapt a corresponding closed/open configuration for the input port, or vice versa.

A pressure gauge is mounted on the shell in fluid communication with the gas enclosure of the hollow container to thereby monitor pressure/volume changes in the gas enclosure. Specifically, these measurements are commensurate with the open/closed configurations of the output port and the corresponding closed/open configurations for the input port. Together, these commensurate measurements indicate liquid flow values passing through the liquid receptacle in a downstream direction from the contractible bag to the output port.

As noted above, included with the present invention is an elastomeric tube that is formed with a lumen which extends from an upstream end to a downstream end. In an unstressed condition of the elastomeric tube, the tube has an open lumen that defines a volume, v_{L}. The upstream end of the tube is connected in fluid communication with the output port of the hollow container. On the other hand, the downstream end is open. Further, a pinch-squeeze mechanism is mounted on the fluid pump for engagement with the elastomeric tube between the upstream end and the downstream end of the tube.

In detail, the pinch-squeeze mechanism includes an upstream pincher and a downstream pincher with a squeeze device that is positioned between the pinchers. In a preferred embodiment, the upstream pincher of the pinch-squeeze mechanism is positioned at the output port of the hollow container and functions to manipulate the open/closed configuration of the output port as disclosed above. In an alternate embodiment, a separate valve is positioned at the output port. The elastomeric tube is then positioned on the squeeze device between the upstream pincher and the downstream pincher.

Operationally, during a machine work cycle the elastomeric tube is positioned on the pinch-squeeze mechanism. In this combination, the function of the squeeze device of the mechanism is to collapse the elastomeric tube and thereby move a liquid volume v_{L} downstream. In this phase of the machine work cycle, the upstream pincher is closed while the downstream pincher is open, causing the squeezed liquid volume to be infused into a patient. Subsequently, the downstream pincher is closed and the upstream pincher is open with the input port of the hollow container also in a closed configuration, causing the squeezed elastomeric tube to rebound from its collapsed condition, thereby exerting a negative pressure on the input port. As a result, a subsequent liquid volume v_{L} is being drawn from the liquid pathway of the hollow container and a measureable reduction in the pressure of the gas in the hollow container. Subsequent to measurement of the reduction in pressure, a control valve is opened, allowing the contractible bag to refill the liquid pathway. During this action the output port of the hollow container is effectively closed by the upstream pincher of the squeeze device. As a result of the staggered open/closed configurations of the pincher and control valves summarized above, there is never an operational configuration where there exists a direct fluid pathway between the contractible bag and the patient.

In detail, during the above-described rebound operation of the squeeze device, the pressure gauge will detect a volume change in the gas enclosure of the hollow container. Specifically, as a volume v_{L} of liquid medicament is being removed from the elastomeric tube in the downstream direction, a volume of liquid Δ(+v_{L}) is introduced into the liquid pathway from the contractible bag. Simultaneously, the gas volume in the gas enclosure is reduced by Δ(−v_{L}). This gas volume change occurs for two reasons. One is because the air pressure in the gas enclosure is maintained less than the atmospheric pressure on the collapsible bag holding the liquid medicament. The other reason is due to the rebound ability of the elastomeric tube.

Subsequently, in a separate action, as the elastomeric tube is rebounding from its squeezed configuration with its downstream end pinched closed and its upstream pincher is opened, a volume v_{L} of liquid medicament is moved from the liquid pathway of the hollow container and into the rebounding elastomeric tube. While the elastomeric tube is being refilled with Δ(+v_{L}), the input port of the hollow container remains closed to prevent liquid flow into the liquid pathway from the collapsible bag. Consequently, the volume of liquid in the liquid pathway is decreased by Δ(−v_{L}). This then causes the volume of the gas enclosure to be increased by Δ(+v_{L}). Thus, in accordance with the present invention, measuring gas volume changes ±Δ(v_{L}) in the gas enclosure can be used to monitor fluid flow through the elastomeric tube for controlling infusions to the patient.

### BRIEF DESCRIPTION OF THE DRAWINGS

The novel features of this invention, as well as the invention itself, both as to its structure and its operation, will be best understood from the accompanying drawings taken in conjunction with the accompanying description, in which similar reference characters refer to similar parts, and in which:

FIG. 1 is a perspective view of a pressure monitor shown incorporated into the system of a medical device for infusing fluid medicaments to a patient;

FIG. 2 is a cross section view of the pressure monitor as would be seen along the line 2-2 in FIG. 1;

FIG. 3 is a functional diagram of components for the control unit for the present invention showing their interconnection required to control an operation of the present invention;

FIG. 4A is a diagram showing the fluid flow pathway through the pressure monitor and elastomeric tube when fluid medicament is being infused to a patient while fluid medicament is being repositioned in the pressure monitor for a subsequent infusion; and

FIG. 4B is a diagram showing the fluid flow pathway through the pressure monitor and elastomeric tube when fluid medicament is being transferred from the pressure monitor and into the elastomeric tube for a subsequent infusion.

### DESCRIPTION OF THE PREFERRED EMBODIMENTS

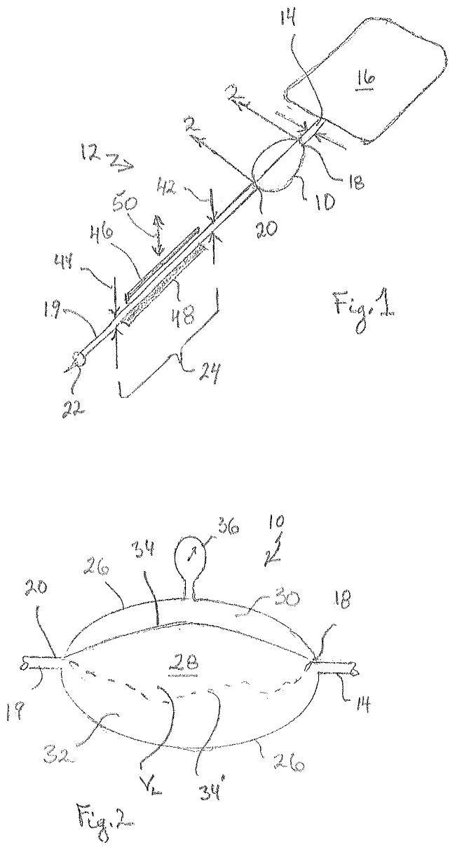

Referring initially to FIG. 1, a pressure monitor for measuring air pressure fluctuations in accordance with the present invention is designated 10. In FIG. 1, the pressure monitor 10 is shown incorporated into a portable infusion system which is generally designated 12. As shown, the system 12 includes a transfer valve 14 which alternatingly connects a contractible bag 16 in fluid communication with an input port 18 of the pressure monitor 10. Further, an elastomeric tube 19 interconnects an output port 20 of the pressure monitor 10 in fluid communication with an infusion implement 22. Between the output port 20 of the pressure monitor 10 and the infusion implement 22, the system 12 includes a fluid pump 24 that is in direct contact with the elastomeric tube 19.

In FIG. 2, the pressure monitor 10 is shown to include a rigid shell 26 which defines a hollow container surrounding a bi-fluid chamber 28. As shown the bi-fluid chamber 28 includes a gas enclosure 30 and a liquid pathway 32 which are separated from each other by a flexible membrane 34. Preferably, the flexible membrane 34 is a non-stretchable material that will move back and forth in the bi-fluid chamber 28 in response to changes in pressures above and below the flexible membrane 34. In its connection with the inside of shell 26, the periphery of the flexible membrane 34 is affixed with a fluid-tight seal to the inside surface of shell 26. Importantly, this connection must not prevent liquid from entering liquid pathway 32 via the input port 18, or from exiting the liquid pathway 32 via the output port 20. Stated differently, although fluid flow is intended on the liquid pathway 32, no liquid medicament will enter the gas enclosure 30.

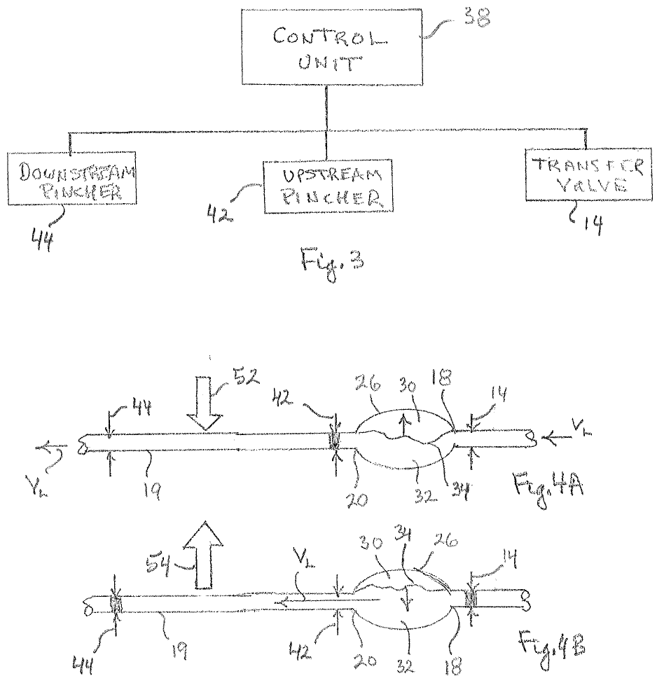

FIG. 2 also shows that a pressure gauge 36 is engaged with the gas enclosure 30 to continuously monitor changes in air pressure/volume inside gas enclosure 30. As disclosed in detail below, these changes in air pressure/volume inside gas enclosure 30 are directly related to trends in the operational fluid flow rate in system 12. Accordingly, a control unit 38 (shown in FIG. 3) is provided to maintain operational parameters for system 12. These parameters are directly influenced by operations of the fluid pump 24 and the transfer valve 14.

Referring again to FIG. 1, it is shown that the fluid pump 24 includes an upstream pincher 42 and a downstream pincher 44. Also, between the upstream pincher 42 and the downstream pincher 44, the fluid pump 24 includes a pinch device that includes a piston 46 and a plate 48. For an operation of the fluid pump 24, a portion of elastomeric tube 19 is placed between the upstream pincher 42 and the downstream pincher 44 and placed between the piston 46 and the plate 48. Further, it is this portion of elastomeric tube 19 that is reciprocally squeezed between the piston 45 and the plate 48 as the piston 46 is moved back and forth against the elastomeric tube 19 in the directions indicated by arrows 50.

Referring to FIG. 3, a functional diagram of control unit 38 is shown connecting it with both the upstream pincher 42 and the downstream pincher 44 of the fluid pump 24. It is also shown that control unit 38 is functionally connected with the transfer valve 14 of the pressure monitor 10. The interaction of these components is best appreciated with reference to FIGS. 4A and 4B.

With reference to FIGS. 4A and 4B, it is to be appreciated that v_{L} is the liquid volume of fluid medicament that is infused to a patient during each machine work cycle. Specifically, this value of v_{L} equals the volume defined when the lumen of the elastomeric tube 19 when the tube 19 is in an unstressed configuration between the upstream pincher 42 and the downstream pincher 44. It is to be also appreciated that a machine work cycle begins when the piston 42 begins to exert a pinch force 52 against the elastomeric tube 19 as shown in FIG. 4A. As shown in FIG. 4B, the machine work cycle subsequently ends when the pinch force 52 is relieved by a rebound force 54, which is created as the elastomeric tube 19 returns to its unstressed configuration.

Operationally, FIG. 4A shows that when the upstream pincher 42 is closed, while the downstream pincher 44 and the transfer valve 14 are both open, the pinch force 52 will force a fluid volume v_{L} from the elastomeric tube 19 in a downstream direction. Simultaneously, as the fluid volume v_{L} is being forced downstream by the pinch force 52 a replacement volume v_{L} of fluid medicament is introduced into the liquid pathway 32 of the shell 26. Specifically, this happens because atmospheric pressure against the contractible bag 16 overcomes a lower pressure in the gas enclosure 30 of the bi-fluid chamber 28 which causes the flexible membrane 34 to move upwardly. It is this overpressure which causes a fluid volume v_{L} to be transferred from the contractible bag 16 into the liquid pathway 32 of the bi-fluid chamber 28.

On the other hand, FIG. 4B shows that when the upstream pincher 42 is opened, while the downstream pincher 44 and the transfer valve 14 are both closed, the elastomeric tube 19 rebounds under the influence of the rebound force 54. As the elastomeric tube 19 returns to its unstressed configuration, a volume v_{L} is drawn from the liquid pathway 32 in the bi-fluid chamber 28 to refill the elastomeric tube 19. This causes the flexible membrane 34 to move downwardly. When considered together, FIGS. 4A and 4B show that during each machine work cycle a volume v_{L} of fluid medicament is transferred downstream from the elastomeric tube 19 for infusion into a patient, while a replacement volume v_{L} is drawn from the contractible bag 16 an into the elastomeric tube 19 via the liquid pathway 32 of the bi-fluid chamber 28.

While the particular fluid flow pressure monitor for a pinch/squeeze pumping action as herein shown and disclosed in detail is fully capable of obtaining the objects and providing the advantages herein before stated, it is to be understood that it is merely illustrative of the of the presently preferred embodiments of the invention and that no limitations are intended to the details of construction or design herein shown other than as described in the appended claims.

What is claimed is:

1. A fluid flow pressure monitor which comprises:
a hollow container having a rigid shell surrounding a bi-fluid chamber having a volume V_{c};
a flexible membrane affixed to the shell inside the bi-fluid chamber to separate the bi-chamber into a gas enclosure and a liquid pathway;
an input port formed on the shell, wherein the input port is adapted to selectively connect a contractible bag containing a volume, V_{b}, of liquid in fluid communication with the liquid pathway of the hollow container, wherein the input port is adapted to alternatingly have a closed/open configuration;
an output port formed on the shell for fluid communication with the liquid pathway, wherein the output port is adapted to alternatingly have an open/closed configuration;
a control unit connected to the input port and to the output port, wherein the control unit is responsive to the open/closed configuration of the output port to correspondingly adapt a closed/open configuration for the input port; and
a pressure gauge mounted on the shell to monitor pressure changes in the gas enclosure commensurate with open/closed configurations of the output port and corresponding closed/open configurations for the input port, as an indication of liquid flow conditions through the liquid receptacle in a downstream direction from the contractible bag to the output port.

2. The pressure monitor of claim 1 further comprising a fluid pump connected with the output port to receive liquid from the contractible bag via the liquid pathway in the hollow container when the output port has an open configuration.

3. The pressure monitor of claim 2 further comprising:
an elastomeric tube formed with a lumen, wherein the elastomeric tube has an upstream end connected in fluid communication with the output port of the hollow container, and a downstream end; and,
a pinch-squeeze mechanism mounted on the fluid pump for engagement with the elastomeric tube.

4. The pressure monitor of claim 3 wherein the pinch-squeeze mechanism includes an upstream pincher, a downstream pincher and a squeeze device positioned there between, wherein an unstressed elastomeric tube has a lumen volume, vL, between the upstream pincher and the downstream pincher of the pinch-squeeze mechanism.

5. The pressure monitor of claim 4 wherein the elastomeric tube is engaged with the pinch-squeeze mechanism and functions to move a liquid volume vL through the elastomeric tube in the downstream direction in response to a collapse of the elastomeric tube caused by the squeeze device of the fluid pump during the closed configuration of the output port of the hollow container while the downstream pincher is open, and to draw a liquid volume vL into the elastomeric tube from the liquid pathway of the hollow container as the elastomeric tube rebounds from the collapse during the open configuration of the output port when the downstream pincher is closed.

6. The pressure monitor of claim 5 wherein during the closed configuration of the output port and the corresponding open configuration of the input port, the volume of the gas enclosure in the hollow container is decreased by Δ(−vL) as a volume of liquid Δ(+vL) is introduced into the liquid pathway from the contractible bag, and further wherein during the open configuration of the output port and the closed configuration of the input port when liquid with volume v_{L} is moved from the liquid pathway and into the rebounding elastomeric tube, the volume of the liquid in the liquid pathway is decreased by Δ(−vL) and the volume of the gas enclosure in the hollow container is increased by Δ(+vL).

7. The pressure monitor of claim 5 wherein the opened and closed configurations of the output port are alternately determined by the action of the upstream pincher of the pinch-squeeze mechanism.

8. The pressure monitor of claim 1 wherein gas pressure changes Δp in the gas enclosure are registered by the pressure gauge and are indicative of liquid volume changes ΔvL in the contractible bag during liquid flow from the contractible bag through the liquid pathway of the hollow container.

9. The pressure monitor of claim 8 wherein the contractible bag of liquid is operationally subject to atmospheric pressure.

10. The pressure monitor of claim 8 wherein for a normal operation a liquid volume change Δvb in the contractible bag is operationally equal to the lumen volume vL in the elastomeric tube between the upstream pincher and the downstream pincher.

11. A system for monitoring liquid flow rate which comprises:
a contractible bag for holding a liquid, wherein the contractible bag is subject to atmospheric pressure;
a fluid pump for sequentially moving liquid from the contractible bag and through an elastomeric tube in a downstream direction, wherein the elastomeric tube has an upstream end connected in fluid communication with the contractible bag and a downstream end; and
a pressure monitor interconnecting the contractible bag with the fluid pump, wherein the pressure monitor includes a flexible membrane separating a gas enclosure from a liquid pathway in a bi-fluid chamber, and wherein measured gas pressure changes, Δp, in the gas enclosure are indicative of a predetermined liquid volume change, ΔvL, in the liquid pathway during liquid flow from the contractible bag through the liquid pathway and into the elastomeric tube.

12. The system of claim 11 further comprising a pinch-squeeze mechanism mounted on the fluid pump for engagement with the elastomeric tube wherein the pinch-squeeze mechanism functions to move a liquid volume vL from the elastomeric tube in the downstream direction in response to a collapse of the elastomeric tube caused by the pinch-squeeze mechanism, and to draw liquid into the elastomeric tube from the liquid pathway of the hollow container as the elastomeric tube is relieved by the pinch-squeeze mechanism and rebounds from the collapse.

13. The pressure monitor of claim 12 wherein the liquid volume change Δvb in the contractible bag is operationally equal to a volume, vL, in a portion of the lumen of the elastomeric tube when the elastomeric tube is engaged with the pinch-squeeze mechanism.

14. The pressure monitor of claim 13 wherein a gas pressure change Δp indicating more than 20% of a normal ΔvL activates a safety alert caused by a restriction of fluid flow from the contractible bag.

15. The pressure monitor of claim 14 wherein the open downstream end is connected with an infusion implement.

16. A method for manufacturing a fluid flow pressure monitor which comprises the steps of:
providing a hollow container having a rigid shell surrounding a bi-fluid chamber;
affixing a flexible membrane inside the bi-fluid chamber to separate the bi-fluid chamber into a gas enclosure and a liquid pathway;
connecting a contractible bag to an input port of the shell, wherein the contractible bag contains a volume, vb, of liquid for fluid communication with the liquid pathway of the hollow container;
connecting an elastomeric tube to an output port of the shell, wherein the elastomeric tube is formed with a lumen, and wherein the elastomeric tube has an upstream end connected in fluid communication with the output port of the hollow container, and an open downstream end; and
engaging the elastomeric tube with a pinch-squeeze mechanism, wherein the pinch-squeeze mechanism includes an upstream pincher, a downstream pincher and a squeeze device positioned there between, wherein an unstressed elastomeric tube has a lumen volume, vL, between the upstream pincher and the downstream pincher of the pinch-squeeze mechanism.

17. The method of claim 16 further comprising the step of establishing a connection between the pinch-squeeze mechanism and the elastomeric tube wherein the squeeze device functions to move a liquid volume vL through the elastomeric tube in the downstream direction by collapsing the elastomeric tube during the closed configuration of the output port of the hollow container, and to draw a liquid volume vL into the elastomeric tube from the liquid pathway of the hollow container as the elastomeric tube rebounds from the collapse.

18. The method of claim 17 further comprising the step of providing a control unit connected with the input port and with the output port of the shell to coordinate an alternating open/closed configuration of the output port of the shell with an alternating closed/open configuration of the input port of the shell, and wherein the control unit is responsive to the closed/open configuration of the input port to move a liquid volume vL from the elastomeric tube in the downstream direction in response to a collapse of the elastomeric tube caused by a squeeze device of the fluid pump, and to draw liquid into the elastomeric tube from the liquid pathway of the hollow container as the elastomeric tube rebounds from the collapse.

19. The method of claim 18 wherein the contractible bag of liquid is subject to atmospheric pressure.

20. The method of claim 19 wherein a gas pressure change Δp indicating more than 20% of a normal ΔvL activates a safety alert caused by a restriction of fluid flow from the contractible bag.
